(19) United States
(12) Patent Application Publication
(10) Pub. No.: US 2014/0271707 A1
(43) Pub. Date: Sep. 18, 2014

(54) METHOD AND COMPOSITION FOR THE TREATMENT OF EXCESS FAT ACCUMULATION

(71) Applicant: DERMAdoctor, Inc., Kansas City, MO (US)

(72) Inventor: Audrey Kunin, Mission Hills, KS (US)

(73) Assignee: DERMAdoctor, Inc., Kansas City, MO (US)

(21) Appl. No.: 14/213,471

(22) Filed: Mar. 14, 2014

Related U.S. Application Data
(60) Provisional application No. 61/798,560, filed on Mar. 15, 2013.

(51) Int. Cl. A61K 8/97; A61Q 19/06; A61Q 19/00; A61Q 19/08; A61K 8/49; A61K 8/67

(52) U.S. Cl. CPC A61K 8/97; A61K 8/4953; A61K 8/678; A61Q 19/00; A61Q 19/08; A61Q 19/06
USPC 424/195.17; 424/728

(57) ABSTRACT

A method and composition is provided for the treatment of excess fat accumulation. In particular, the composition includes a cosmetic base containing: emollients, emulsifiers, thickeners, preservatives, phytoecdysteroids blend, esthetic enhancers, diuretics, constrictors, metabolic enhancers, adipocyte inhibitors, antioxidants, and botanical extracts. The composition is applied to those areas of the body where the user desires the reduction of excess fat, including the eye and thigh areas.

### CROSS-REFERENCE TO RELATED APPLICATIONS

This application claims priority to U.S. Provisional Patent Application Ser. No. 61/798,560, filed on Mar. 15, 2013, entitled “Method and Composition for the Treatment of Excess Fat Accumulation,” currently pending, the entire disclosure of which is incorporated herein by reference.

### TECHNICAL FIELD

The present application relates to skin care compositions. More specifically, skin care compositions are described that treat the accumulation of excess fat underneath the surface of the skin.

### BACKGROUND OF THE INVENTION

According to the National Institutes of Health, cellulite is fat that is deposited in pockets just below the surface of the skin. It occurs around the hips, thighs, and buttocks. Because it is very close to the surface of the skin, cellulite leads to a dimpled appearance and makes the thighs and buttocks look like cottage cheese.

Cellulite may be more visible than fat deeper in the body. Even thin people can have cellulite, because we all have layers of fat just below the surface of the skin. Collagen fibers that connect fat to the skin may stretch, break down, or pull tight, allowing the fat cells to bulge out. This creates the rippled look of cellulite.

Although many dermatologists and cosmetic surgeons recognize cellulite as a legitimate problem that patients seek to have them “cure,” most of the medical community does not view cellulite as a disorder. Instead, it is considered a normal condition of many women and some men.

At some point in their lives, most women, and some men, are bothered to a degree by cellulite. Although its name may make it sound like a medical condition, cellulite is nothing more than normal fat beneath the skin. The fat appears bumpy because it pushes against the connective tissue beneath the skin, causing the skin above it to pucker.

Although the cellulite is not harmful, many people are willing to go to great lengths to get rid of cellulite, creating a big market for cellulite creams and other treatments promising smooth thighs.

Excess fat accumulation may also occur in the lid and under eye area. As you age, the tissue structures and muscles supporting your eyelids weaken. The skin may start to sag, and fat that is normally confined to the area around the eye (orbit) can migrate forward below your eyes. In some instances, the space below your eyes can accumulate fluid, making the under-eye area appear puffy or swollen.

Bags under eyes are usually a cosmetic concern and do not require specific treatment. Depending on the cause of the swelling, treatments, such as applying cold compresses or sleeping with your head raised, can help reduce or eliminate puffy eyes. Medical and surgical treatments are available if a person is concerned about the appearance of under-eye swelling. Treatment may not be covered by medical insurance if it is done solely to improve your appearance.

Skin treatments traditionally done to treat wrinkles, such as laser resurfacing or chemical peels, may improve skin tone and tighten the skin. This may lessen the appearance of under-eye swelling and improve any discoloration. However, these types of treatments, if applied incorrectly, can also result in damage to skin and painful burns.

Cellulite and excess fat can also be addressed by surgery. For example, blepharoplasty, a form of eyelid surgery, is an option to remove bags under eyes. During blepharoplasty, the surgeon cuts just below the lashes in a patient's eye in the natural crease or inside the lower lid. The surgeon removes excess fat and sagging skin. Depending on where the initial incisions are made, stitches may follow the lower lid's natural crease or be placed inside the lower eyelid. In addition to correcting bags under eyes, blepharoplasty can also repair: (i) baggy or puffy upper eyelids; (ii) excess skin of the upper eyelid that interferes with your vision; (iii) droopy lower eyelids, which may cause white to show below the iris—the colored part of the eye; and (iv) excess skin on lower eyelids.

Additionally, more evasive surgical techniques such as liposuction wherein large quantities of fat are removed by a cannula (a hollow tube) and aspirator (a suction device) may be used. However, surgical techniques may require significant recovery time and have possible painful side effects.

Alternatively, there are a variety of topical therapies available to reduce the appearance of cellulite and the puffiness of the under eye area. These include body wraps and laser treatments. Also, creams and lotions with diuretics, emollients and moisturizers are available to make the skin feel smoother and perhaps reduce the appearance of dimples through the diuretic-effect of removing fluid build-up.

Generally, these topical therapies produce a temporary effect and do not reduce the amount of fat in fat cells or reduce the amount of fat build-up. For example, endermologie has been FDA approved for cellulite/inch reduction for over 15 years. Endermologie involves a technician manipulating a patented endermologie suction device, which performs vacuum-like suction to the affected areas, usually the arms, thighs, buttocks and the belly. The device gently kneads the skin in these areas, which is supposed to help stimulate circulation, as well as to encourage “lymphatic drainage”. Lymphatic drainage is where the local lymph system is relieved of toxins and expelled from the body. The process pushes fluid out from between fat lobes, “wrings out” the actual fat lobe, and then lays them back down in a flat, even layer, much like a steam roller smoothes out jumbled gravel into a nice, smooth road. This is supposed to help cellulite disappear, based on the somewhat popular theory that cellulite is caused by both poor circulation and toxin build-up.

However, endermologie is a progressive treatment that requires a patient commit to having a treatment once or twice a week over the course of 14 to 20 sessions. After the initial treatments, patients need to continue with treatments to maintain results. So it is important a patient commit to the treatment performed regularly, usually weekly, in order to maintain results.

There are several topicals that promise to reduce the appearance of the fat bags, excess fat and puffiness around the eye area, or to reduce wrinkles too. However, many topical solutions do not reduce the actual fat cells, do not reduce the amount of fat in the fat cells and do not increase the production of collagen and elastin.

Therefore, it would be beneficial to have a method and composition for the treatment of excess fat accumulation that contains an effective amount of medication for such treatment. It would be further beneficial to have a treatment for cellulite that does not require surgery and has little to no harmful side effects.

### SUMMARY OF THE INVENTION

Topical skin care compositions for treatment of excess accumulation of adipose tissue (fat) and loss of skin firmness and tone are disclosed. The disclosed skin care compositions have the unique ability to reduce the amount of lipid accumulation in adipocytes (fat cells), degrade existing adipocytes, and increase firmness of the skin through increased collagen and elastin production. The reduction of the adipocyte volume reduces the appearance of dimpling and the increase in collagen and elastin lift and firm areas that previously sagged and drooped due to a loss of collagen and elastin.

In particular, a topical skin care composition may be comprised of a cosmetic base containing: emollients, emulsifiers, thickeners, preservatives, phytoecdysteroids blend, esthetic enhancers, diuretics, constrictors, metabolic enhancers, adipocyte inhibitors, antioxidants, and botanical extracts.

In one non-limiting illustration, the composition contains: aqua (water), isopropyl myristate, glycerin, stearic acid, cetyl alcohol, cetearyl alcohol, propanediol, euglena gracilis extract, petrolatum, dimethicone, ppg-3 benzyl ether myristate, cyclopentasiloxane, glyceryl stearate, peg-100 stearate, dicetyl phosphate, ceteth-10 phosphate, cyclohexasiloxane, hedera helix (ivy) extract, zizyphus jujuba seed extract, dihydromyricetin, caffeine, phytoecdysteroids, cynara scolymus (artichoke) bud extract, decyl glucoside, levan, myrica cerifera (bayberry) fruit extract, glaucium flavum leaf extract, carbomer, sodium hydroxide, disodium EDTA, phenethyl alcohol, glucose, methyl xanthine, theobromine, paraxanthine, phenoxyethanol, caprylyl glycol, ethylhexylglycerin, and hexylene glycol.

In another non-limiting illustration, the composition of the present invention contains: aqua (water), cyclopentasiloxane, caprylic/capric triglyceride, yeast amino acids, butyl methoxydibenzoylmethane, butylene glycol, glycerin, polysilicone-11, aminobutyric acid, barium sulfate, cetyl alcohol, stearic acid, propanediol, euglena gracilis extract, glyceryl stearate, peg-100 stearate, tricalcium phosphate, dimethicone, steareth-20, panax ginseng root extract, portulaca oleracea extract, centella asiatica extract, caffeine, morinda citrifolia extract, zizyphus jujuba seed extract, hydroxyethylcellulose, acetyl dipeptide-1 cetyl ester, phytoecdysteroids, glaucium flavum leaf extract, sodium stearoyl glutamate, tocopheryl acetate, cyclohexasiloxane, xanthan gum, sorbitan laurate, disodium EDTA, phenoxyethanol, methylparaben, ethylparaben, propylparaben, butylparaben, and diazolidinyl urea.

Other and further objects of the invention, together with the features of novelty appurtenant thereto, will appear in the course of the following description.

### FIGURES

FIG. 1 illustrates the use of a composition of the present invention on a patient after two weeks.

FIG. 2 illustrates the use of a composition of the present invention on a patient after four weeks.

FIG. 3 illustrates the use of a composition of the present invention on a patient after eight weeks.

FIG. 4 illustrates the use of a composition of the present invention on a patient from the panelist's pictures shown in FIG. 4 with regard to lift: red horizontal line is always same length and position, green vertical line from red line to upper eyelid area (measuring lift of upper eyelid/brow).

### DETAILED DESCRIPTION OF THE INVENTION

There is provided herein a composition for the treatment of excess fat accumulation.

Topical skin care compositions for treatment of excess accumulation of adipose tissue (fat) and loss of skin firmness and tone are disclosed. The disclosed skin care compositions have the unique ability to reduce the amount of lipid accumulation in adipocytes (fat cells), degrade existing adipocytes, and increase firmness of the skin through increased collagen and elastin production. The reduction of the adipocyte volume reduces the appearance of dimpling and the increase in collagen and elastin lift and firm areas that previously sagged and drooped due to a loss of collagen and elastin.

In particular, a topical skin care composition is provided that may include a base mixture, emollients, emulsifiers, thickeners, preservatives, phytoecdysteroids blend, esthetic enhancers, diuretics, constrictors, metabolic enhancers, adipocyte inhibitors, antioxidants, and botanical extracts.

The topical skin care composition may comprise a base. Such a mixture may comprise water, sodium hydroxide, phenethyl alcohol, butyl methoxydibenzoylmethane, and barium sulfate. The base may comprise from about 30.0% to about 98.0% of the formulation by weight.

The topical skin care composition may comprise an emollient. Examples of emollients that are suitable, among others, include glycerin, cetearyl alcohol, cetyl alcohol, cyclopentasiloxane, glyceryl stearate, dimethicone, caprylyl glycol, ethylhexylglycerin, isopropyl myristate, glucose, petrolatum, PPG-3 benzyl ether myristate, cyclohexasiloxane, levan, caprylic/capric triglyceride, yeast amino acids, butylene glycol, aminobutyric acid, panax ginseng root extract, portulaca oleracea extract, centella asiatica extract, and acetyl dipeptide-1 cetyl ester. An emollient may comprise from about 1.0% to about 20.0% of the formulation by weight.

The topical skin care composition may comprise an emulsifier. Examples of emulsifiers that are suitable components for the composition include among others stearic acid, PEG-100 stearate, hexylene glycol, dicetyl phosphate, ceteth-10 phosphate, decyl glucoside, steareth-20, hydroxyethylcellulose, sodium stearoyl glutamate, xanthan gum, sorbitan laurate. One or more emulsifiers may comprise from about 1.0% to about 10.0% of the formulation by weight.

The topical skin care composition may include a thickener. Examples of suitable thickeners include carbomer, propanediol, Polysilicone-11, and disodium EDTA. One or more thickeners may comprise from about 0.0% to about 4.0% of the formulation by weight.

The topical skin care composition may include preservatives such as phenoxyethanol, methylparaben, ethylparaben, propylparaben, butylparaben, and diazolidinyl urea. One or more preservative may comprise from about 0.0% to about 3.0% of the formulation by weight.

The topical skin care composition may include a phytoecdysteroid blend which can be comprised of one or more of the following: phytoecdysteroids, euglena gracilis extract, zizyphus jujuba seed extract, glaucium flavum leaf, myrica cerifera extract, and cynara scolymus (artichoke) bud extract. The phytoecdysteroid blend may comprise from about 0.1% to about 6.0% of the formulation by weight.

The topical skin care composition may be comprised of esthetic enhancers such as dihydromyricetin, levan, tricalcium phosphate, yeast amino acids, and morinda citrifolia extract. One or more esthetic enhancers may comprise from about 0.0% to about 6.0% of the formulation by weight.

The topical skin care composition may be comprised of a diuretic such as caffeine. One or more diuretics may comprise from about 0.1% to about 3.0% of the formulation by weight.

The topical skin care composition may be comprised of a constrictor such as hedera helix (ivy) extract. One or more constrictors may comprise from about 0.0% to about 1.0% of the formulation by weight.

The topical skin care composition may be comprised of a metabolic enhancer such as myrica cerifera (bayberry) fruit extract and glaucium flavum leaf extract. One or more metabolic enhancers may comprise from about 0.0% to about 1.0% of the formulation by weight.

The topical skin care composition may be comprised on an antioxidant such as tocopheryl acetate. One or more antioxidants may comprise from about 0.0% to about 1.0% of the formulation by weight.

The topical skin care composition may be comprised of adipocyte inhibitors such as zizyphus jujuba seed extract, myrica cerifera (bayberry) fruit extract, glaucium flavum leaf extract, cynara scolymus (artichoke) bud extract, methyl xanthine, theobromine, and paraxanthine. One or more adipocyte inhibitors may comprise from about 0.02% to about 5.0% of the formulation by weight.

The topical skin care composition may be comprised of a botanical extracts such as euglena gracilis extract, myrica cerifera (bayberry) fruit extract, glaucium flavum leaf extract, zizyphus jujuba seed extract, glaucium flavum leaf extract, and morinda citrifolia extract. One or more botanical extracts may comprise from about 0.0% to about 5.0% of the formulation by weight.

In one non-limiting illustration, the composition contains:
a base: aqua (water), sodium hydroxide (stabilizer), phenethyl alcohol (masking)
emollients: glycerin, cetearyl alcohol, cetyl alcohol, cyclopentasiloxane, glyceryl stearate, dimethicone, caprylyl glycol, ethylhexylglycerin, isopropyl myristate, glucose, petrolatum, ppg-3 benzyl ether myristate, cyclohexasiloxane, levan (skin protectant)
emulsifiers: stearic acid, peg-100 stearate, hexylene glycol, dicetyl phosphate, ceteth-10 phosphate, decyl glucoside
thickeners: carbomer, propanediol, disodium EDTA
preservatives: phenoxyethanol
Phytoecdysteroid Blend: phytoecdysteroids, euglena gracilis extract
esthetic enhancers: dihydromyricetin, levan (skin protectant)
diuretics: Caffeine
constrictors: hedera helix (ivy) extract
metabolic enhancers: myrica cerifera (bayberry) fruit extract, glaucium flavum leaf extract
adipocyte inhibitors: zizyphus jujuba seed extract, myrica cerifera (bayberry) fruit extract, glaucium flavum leaf extract, cynara scolymus (artichoke) bud extract, methyl xanthine, theobromine, paraxanthine
antioxidants: tocopheryl acetate
botanical extracts: euglena gracilis extract, hedera helix (ivy) extract, myrica cerifera (bayberry) fruit extract, glaucium flavum leaf extract, zizyphus jujuba seed extract, cynara scolymus (artichoke) bud extract

In another non-limiting illustration, the composition contains:
a base: aqua (water), butyl methoxydibenzoylmethane (uv absorber/filter), barium sulfate (opacifier)
emollients: glycerin, cetyl alcohol, cyclopentasiloxane, glyceryl stearate, dimethicone, cyclohexasiloxane, caprylic/capric triglyceride, yeast amino acids, butylene glycol, aminobutyric acid, panax ginseng root extract, portulaca oleracea extract, centella asiatica extract, acetyl dipeptide-1 cetyl ester,
emulsifiers: stearic acid, peg-100 stearate, steareth-20, hydroxyethylcellulose, sodium stearoyl glutamate, xanthan gum, sorbitan laurate
thickeners: propanediol, disodium EDTA, polysilicone-11
preservatives: phenoxyethanol, methylparaben, ethylparaben, propylparaben, butylparaben, diazolidinyl urea
Phytoecdysteroid Blend: zizyphus jujuba seed extract, euglena gracilis extract, glaucium flavum leaf, myrica cerifera extract, cynara scolymus (artichoke) bud extract
esthetic enhancers: tricalcium phosphate, yeast amino acids, morinda citrifolia extract,
diuretics: caffeine
metabolic enhancers: myrica cerifera (bayberry) fruit extract, glaucium flavum leaf extract
adipocyte inhibitors: zizyphus jujuba seed extract, glaucium flavum leaf extract
antioxidants: tocopheryl acetate
botanical extracts: euglena gracilis extract, myrica cerifera (bayberry) fruit extract, glaucium flavum leaf extract, zizyphus jujuba seed extract, and morinda citrifolia extract.

An example of the skin care formulation comprises:

| Example Formulation #1 | % Weight |
|---|---|
| Aqua (Water) (and) Cyclopentasiloxane (and) | 35.00 |
| Polysilicone-11 (and) Aminobutyric Acid (and) | |
| Barium Sulfate (and) Panax Ginseng Root | |
| Extract (and) Portulaca Oleracea Extract (and) | |
| Centella Asiatica Extract | |
| Aqua (Water) | QS to 100 |
| Caprylic/Capric Triglyceride | 4.25 |
| Yeast Amino Acids (and) Tricalcium Phosphate | 5.00 |
| (and) Morinda Citrifolia Extract | |
| Butylene Glycol (and) Aqua (Water) (and) Sorbitan | 4.00 |
| Laurate (and) Hydroxyethylcellulose (and) Acetyl | |
| Dipeptide-1 Cetyl Ester | |
| Caffeine (and) Glaucium Flavum Leaf Extract | 3.00 |
| (and) Euglena Gracilis Extract (and) Aqua (Water) | |
| Butyl Methoxydibenzoylmethane | 2.25 |
| Glyceryl Stearate (and) PEG-100 Stearate | 2.10 |
| Glycerin (and) Aqua (Water) | 2.00 |
| Stearic Acid | 1.75 |
| Cetyl Alcohol | 1.50 |
| Propanediol (and) Aqua (Water) (and) Zizyphus | 2.00 |
| Jujuba Seed Extract (and) Phytoecdysteroids | |
| Phenoxyethanol (and) Methylparaben (and) | 1.10 |
| Butylparaben (and) Ethylparaben (and) Propylparaben | |
| Dimethicone | 0.50 |
| Cyclopentasiloxane (and) Cyclohexasiloxane | 0.50 |
| Sodium Stearoyl Glutamate | 0.30 |
| Tocopheryl Acetate | 0.25 |
| Steareth-20 | 0.25 |
| Diazolidinyl Urea | 0.15 |
| Xanthan Gum | 0.05 |
| Caffeine | 0.05 |
| Disodium EDTA | 0.01 |

Another example of the formulation comprises:

| Example Formulation #2 | % Weight |
|---|---|
| Aqua (Water) | QS to 100 |
| Isopropyl Myristate | 5.500 |
| Stearic Acid | 4.500 |
| Glycerin (and) Aqua (Water) | 4.000 |
| caffeine (and) Glaucium Flavum Leaf Extract | 3.000 |
| (and) Euglena Gracilis Extract (and) Aqua (Water) | |
| Cetearyl Alcohol(and) Dicetyl Phosphate (and) | 2.000 |
| Ceteth-10 Phosphate | |
| Cetyl Alcohol | 1.750 |
| Propanediol (and) Aqua (Water) (and) Zizyphus | 2.000 |
| Jujuba Seed Extract (and) Phytoecdysteroids | |
| (NF) Petrolatum | 1.000 |
| Aqua (Water) (and) Glycerin (and) Hedera Helix | 0.750 |
| (Ivy) Extract | |
| Glyceryl Stearate (and) PEG-100 Stearate | 0.750 |
| Dimethicone | 0.750 |
| Phenoxyethanol (and) Caprylyl Glycol (and) | 0.500 |
| Ethylhexylglycerin (and) Hexylene Glycol | |
| PPG-3 Benzyl Ether Myristate | 0.500 |
| Cyclopentasiloxane (and) Cyclohexasiloxane | 0.500 |
| Aqua (Water) (and) Levan (and) Cynara Scolymus | 0.250 |
| (Artichoke) Bud Extract (and) Decyl Glucoside | |
| (and) Phenethyl Alcohol (and) Glucose (and) | |
| Caffeine (and) Theobromine (and) MethylXanthine | |
| (and) Paraxanthine | |
| Carbomer | 0.100 |
| Sodium Hydroxide | 0.100 |
| Disodium EDTA | 0.050 |
| Myrica Cerifera (Bayberry) Leaf Extract (and) | 0.050 |
| Dihydromyricetin | |

An aspect of this formulation is that upon being applied to the area of the skin where treatment is desired, the combination of several ingredients that inhibit fat accumulation within fat cells, inhibit the formation of new fat cells, inhibit pre-adipocyte differentiation, decrease the amount of fat contained in fat cells, stimulate degradation of existing fat, increase the mitochondrial energy, trigger fragmentation of lipid droplets, promote vasoconstriction and anti-cellulite effects, increase the production of elastin and collagen to work with the included phytoecdysteroid blend. This blend consists of botanical chemicals that are bio-identical to the ecdysteroid hormone in crustaceans and arthropods. Phytoecdysteroids activate the gene expression of the dermal-epidermal junction (DEJ) components and strengthen the intercellular unions. This increases skin firmness and elasticity.

### PDT EYE Panelist Measurements Baseline and Each Check in Period

TABLE 1
Measurements from the panelist's pictures shown
in FIG. 4 with regard to lift: red horizontal line is always
same length and position, green vertical line from red
line to upper eyelid area (measuring lift of upper eyelid/brow)

| Panel ID | Time | Inches | % change* |
|---|---|---|---|
| 41072 | initial | 0.234 | |
| 41101 | wk 4 | 0.344 | 47% |
| 41072 | initial | 0.234 | |
| 41114 | wk 6 | 0.344 | 47% |
| 41072 | initial | 0.234 | |
| 41128 | wk 8 | 0.391 | 67% |
| 41072 | initial | 5.9 | |
| 41101 | wk 4 | 8.7 | 47% |
| 41072 | initial | 5.9 | |
| 41114 | wk 6 | 8.7 | 47% |
| 41072 | initial | 5.9 | |
| 41128 | wk 8 | 9.9 | 68% |

| | | |
|---|---|---|
| period | inches | mm |

| | | | |
|---|---|---|---|
| baseline | 15/64 | 0.234 | 5.9 |
| wk 4 | 22/64 | 0.344 | 8.7 |
| wk 6 | 22/64 | 0.344 | 8.7 |
| wk 8 | 25/64 | 0.391 | 9.9 |

*The percentages are a numerical value of the visual markings and improvements seen in the panelist picture.

### Panelist Measurements (Inches)

TABLE 2
Measurements from the testing of the present invention on the thigh of panelist participants.

| | | | week 1 | week 2 | week 3 | week 4 | week 5 | week 6 | | week 8 | week 9 |
|---|---|---|---|---|---|---|---|---|---|---|---|
| | | baseline | 23- | 29- | 5- | 12- | 19- | 26- | week 7 | 7- | 15- |
| Panelist | Thigh | 15-May | May | May | Jun | Jun | Jun | Jun | 3-Jul | Aug | Aug |
| MH | Right | 23.50 | 23.00 | 23.00 | 23.00 | 23.00 | 22.75 | 22.25 | 22.50 | 22.50 | 22.50 |
| MH | Left | 23.00 | 22.88 | 22.50 | 22.50 | 22.50 | 22.50 | 22.50 | 22.00 | 22.00 | 22.00 |
| BK | Right | 26.67 | 0.00 | 25.63 | 25.17 | 25.96 | 25.58 | 26.29 | 0.00 | 0.00 | 26.00 |
| BK | Left | 26.29 | 0.00 | 25.71 | 26.00 | 25.96 | 26.79 | 26.50 | 0.00 | 0.00 | 26.13 |
| M | Right | 25.38 | 25.38 | 25.50 | 0.00 | 25.54 | 0.00 | 25.88 | 25.58 | 25.25 | 25.92 |
| M | Left | 25.25 | 25.25 | 25.00 | 0.00 | 25.42 | 0.00 | 25.79 | 25.46 | 25.38 | 25.71 |
| D | Right | 30.54 | 0.00 | 29.92 | 30.08 | 30.08 | 30.13 | 0.00 | 30.38 | 30.21 | 30.42 |
| D | Left | 30.58 | 0.00 | 29.63 | 29.88 | 29.88 | 29.71 | 0.00 | 30.00 | 29.38 | 29.79 |
| MP | Right | 24.21 | 24.42 | 24.33 | 24.63 | 25.04 | 0.00 | 24.75 | 25.46 | 0.00 | 25.25 |
| MP | Left | 25.04 | 24.33 | 24.71 | 24.63 | 25.42 | 0.00 | 25.21 | 25.83 | 0.00 | 25.00 |

### Difference in Thigh Measurement (Inches) Compared to Baseline

TABLE 3
Measurements from the testing of the present
invention on the thigh of panelist participants.

| Panelist | Thigh | 1 | week 2 | 3 | week 4 | 5 | week 6 | 7 | week 8 | week 9 |
|---|---|---|---|---|---|---|---|---|---|---|
| MH | Right | | −0.50 | | −0.50 | | −1.25 | | −1.00 | −1.00 |
| MH | Left | | −0.50 | | −0.50 | | −0.50 | | −1.00 | −1.00 |
| BK | Right | | −1.00 | | −0.70 | | −0.38 | | no meas. | −0.67 |
| BK | Left | | −0.60 | | −0.30 | | 0.21 | | no meas. | −0.17 |
| M | Right | | 0.10 | | 0.20 | | 0.50 | | −0.13 | 0.54 |
| M | Left | | −0.30 | | 0.20 | | 0.54 | | 0.13 | 0.46 |
| D | Right | | −0.60 | | −0.50 | | no meas. | | −0.33 | −0.13 |
| D | Left | | −1.00 | | −0.70 | | no meas. | | −1.21 | −0.79 |
| MP | Right | | 0.10 | | 0.80 | | 0.54 | | no meas. | 1.04 |
| MP | Left | | −0.30 | | 0.40 | | 0.17 | | no meas. | −0.04 |

Panelists used the formulation as directed. Thigh measurements were taken with a standard tape measure. Readings were taken 3 times at the same area on each thigh. The Average Measurement was recorded for the Right Thigh and then the Left Thigh. Table 2 shows the average for the Right Thigh and for the Left Thigh for each panelist at baseline and at each check in period. Table 3 shows the change in Thigh Measurement compared to the Baseline Measurement for each panelist.

From the foregoing, it will be seen that this invention is one well adapted to attain all ends and objects hereinabove set forth together with the other advantages which are obvious and which are inherent to the structure. It will be understood that certain features and subcombinations are of utility and may be employed without reference to other features and subcombinations. This is contemplated by and is within the scope of the claims. Since many possible embodiments may be made of the invention without departing from the scope thereof, it is to be understood that all matter herein set forth is to be interpreted as illustrative, and not in a limiting sense.

What is claimed is:

1. A skin care composition for the treatment of excess fat accumulation of a user's body comprising:
a cosmetic base containing therein,
(a) an emollient;
(b) an emulsifier;
(c) an adipocyte inhibitor;
(d) a metabolic enhancer;
(e) a diuretic; and
(f) a phytoecdysteriod blend.

2. The composition of claim 1 further comprising an esthetic enhancer.

3. The composition of claim 1 further comprising a constrictor.

4. The composition of claim 1 further comprising a botanical extract.

5. The composition of claim 1 further comprising a thickener.

6. The composition of claim 1 further comprising a preservative.

7. A method for the treatment of excess fat accumulation on a user's body by applying at the area requiring treatment a skin care composition comprising:
a cosmetic base containing therein,
(a) an emollient;
(b) an emulsifier;
(c) an adipocyte inhibitor;
(d) a metabolic enhancer;
(e) a constrictor;
(f) a diuretic; and
(g) a phytoecdysteriod blend.

8. The method of claim 7 wherein the composition further comprises an esthetic enhancer.

9. The method of claim 7 wherein the composition further comprises a constrictor.

10. The method of claim 7 wherein the composition further comprises a botanical extract.

11. The method of claim 7 wherein the composition further comprises an antioxidant.

12. The method of claim 7 wherein the composition further comprises a thickener.

13. The method of claim 7 wherein the composition further comprises a preservative.

14. A skin care composition for the treatment of excess fat accumulation of a user's body comprising:
a cosmetic base containing therein,
(a) an emollient;
(b) an emulsifier;
(c) an adipocyte inhibitor;
(d) a metabolic enhancer;
(e) a diuretic;
(f) a phytoecdysteriod blend;
(g) an esthetic enhancer;
(h) a botanical extract;
(i) an antioxidant;
(j) a thickener; and
(k) a preservative.
